(19) United States
(12) Patent Application Publication
(10) Pub. No.: US 2021/0290325 A1
(43) Pub. Date: Sep. 23, 2021

(54) MEDICAL DEVICE, MEDICAL DEVICE COMPONENT AND MASTER-SLAVE SYSTEM

(71) Applicant: SONY CORPORATION, TOKYO (JP)

(72) Inventors: HIROYUKI SUZUKI, TOKYO (JP); RYUTA HORIE, TOKYO (JP); KATSUFUMI SUGIMOTO, TOKYO (JP)

(21) Appl. No.: 17/250,570

(22) PCT Filed: Jul. 31, 2019
(86) PCT No.: PCT/JP2019/030089

§ 371 Date: Feb. 3, 2021

(30) Foreign Application Priority Data
Aug. 13, 2018 (JP) 2018-152505

(51) Int. Cl. A61B 34/37

(52) U.S. Cl. CPC A61B 34/37

(57) ABSTRACT

There is provided a medical device including: a medical instrument (110) that is connected to a drive unit (150) via a power transmission mechanism and driven by the drive unit; a first structural member (120) having a first hollow portion through which the power transmission mechanism (152, 154) is inserted; a second structural member (130) having a second hollow portion through which the first structural member is inserted; a third structural member (140) that couples the first structural member and the second structural member; and a sensor unit (160) that measures vibrations related to a space between an outer wall of the first structural member, an inner wall of the second structural member, and the third structural member, which is on a medical instrument side.

### TECHNICAL FIELD

The present disclosure relates to a medical device, a medical device component, and a master-slave system.

### BACKGROUND ART

In recent years, as a surgical operation system used when endoscopic surgery is performed, a master-slave type system (hereinafter, also referred to as a master-slave system) that enables an approach to an affected part without making a large incision in a patient's body has become known. In such a master-slave system, an operator (a user) such as a doctor operates a master device including an input interface, and a slave device including a medical instrument such as a forceps or tweezers is remotely operated in accordance with the operation of the master device by the operator. The slave device is configured as, for example, an arm device having a distal end for holding the medical device, and a position or attitude of the medical device inside an abdominal cavity can be changed.

In such a master-slave system, in a case in which no contact state between the patient and the medical device is detected, the operator may be unaware that the medical device is in contact with the patient and damage the patient's tissue. Therefore, a technique has been developed in which a contact state between a patient and a medical device is detected on a slave device side and feedback of the contact state is performed on a master device side to an operator. The technique includes, for example, a technique in which a sensor for measuring information on the contact state between the patient and the medical device is provided in the slave device, the information on the contact state measured by the sensor is transmitted to the master device, and a tactile sensation such as vibration is presented on the master device side to the operator. In relation to this technique, for example, the following PTL 1 discloses a technique of detecting a contact state between a patient and a medical device with higher sensitivity and presenting a tactile sensation to an operator.

### CITATION LIST

### Patent Literature

[PTL 1]
JP 2016-214715A

### SUMMARY

### Technical Problem

However, in the above technique, in addition to the information regarding the contact state between the patient and the medical device, vibrations and driving sounds of a motor of the slave device, vibrations from an installation location, and vibrations unrelated to contacts such as noise from the surroundings (hereinafter also referred to as vibration noise) are cross-talked and measured by the sensor. Since such vibration noise is included in the vibrations presented to the operator as the tactile sensation by the master device, it may adversely affect the operation of the master device by the operator.

Therefore, the present disclosure proposes a new and improved medical device, a medical device component, and a master-slave system by which vibration noise transmitted to a sensor can be reduced.

### Solution to Problem

According to the present disclosure, there is provided a medical device including: a medical instrument that is connected to a drive unit via a power transmission mechanism and driven by the drive unit; a first structural member having a first hollow portion through which the power transmission mechanism is inserted; a second structural member having a second hollow portion through which the first structural member is inserted; a third structural member that couples the first structural member to the second structural member; and a sensor unit that measures vibrations related to a space between an outer wall of the first structural member, an inner wall of the second structural member, and the third structural member, which is on a medical instrument side.

Also, according to the present disclosure, there is provided a medical device component including: a second structural member having a second hollow portion through which a first structural member of a medical device is inserted, the medical device including a medical instrument that is connected to a drive unit via a power transmission mechanism and driven by the drive unit, and the first structural member having a first hollow portion through which the power transmission mechanism is inserted; a third structural member that couples the first structural member to the second structural member; and a sensor unit that measures vibrations related to a space between an outer wall of the first structural member, an inner wall of the second structural member, and the third structural member, which is on a medical instrument side.

Also, according to the present disclosure, there is provided a master-slave system including: a medical device; a slave device provided with the medical device; and a master device used to operate the slave device, in which the medical device includes: a medical instrument that is connected to a drive unit via a power transmission mechanism and driven by the drive unit; a first structural member having a first hollow portion through which the power transmission mechanism is inserted; a second structural member having a second hollow portion through which the first structural member is inserted; a third structural member that couples the first structural member to the second structural member; and a sensor unit that measures vibrations related to a space between an outer wall of the first structural member, an inner wall of the second structural member, and the third structural member, which is on a medical instrument side.

### Advantageous Effects of Invention

As described above, according to the present disclosure, it is possible to reduce vibration noise transmitted to a sensor. Also, the above effects are not necessarily intended as limiting and, in addition to or in place of the above effects, any of the effects presented herein or any other effects that can be ascertained from the present specification may be achieved.

### BRIEF DESCRIPTION OF DRAWINGS

FIG. 1 is an explanatory diagram showing a schematic configuration of a master-slave system according to an embodiment of the present disclosure.

FIG. 2 is an explanatory diagram showing an example of a slave device according to the embodiment.

FIG. 3 is an explanatory diagram showing an example of a master device according to the embodiment.

FIG. 4 is a perspective view showing an external configuration of a medical device according to the embodiment.

FIG. 5 is a cross-sectional view along line I-I of the medical device according to the embodiment.

FIG. 6 is a cross-sectional view along line II-II of the medical device according to the embodiment.

FIG. 7 is a cross-sectional view along line III-III of the medical device according to the embodiment.

FIG. 8 is a cross-sectional view along line IV-IV of the medical device according to the embodiment.

FIG. 9 is a simplified diagram showing a medical device according to a comparative example.

FIG. 10 is a simplified diagram showing the medical device according to the embodiment of the present disclosure.

FIG. 11 is a cross-sectional view along line V-V of the medical device according to the embodiment.

FIG. 12 is a block diagram showing a functional configuration example of a control device according to the embodiment.

FIG. 13 is an explanatory diagram showing a first modified example according to the embodiment.

FIG. 14 is an explanatory diagram showing a second modified example according to the embodiment.

FIG. 15 is a cross-sectional view along line VI-VI of the medical device according to the embodiment.

FIG. 16 is an explanatory diagram showing a third modified example according to the embodiment.

FIG. 17 is a block diagram showing an example of a hardware configuration of the control device according to the embodiment.

### DESCRIPTION OF EMBODIMENTS

Preferred embodiments of the present disclosure will be described in detail below with reference to the accompanying figures. In addition, in the present specification and the figures, components having substantially the same functional configuration will be denoted by the same reference numerals, and thus repeated descriptions thereof will be omitted.

Also, the description will be given in the following order.

### 1. Overview

2. Embodiments of present disclosure
2-1. Configuration of master-slave system
2-2. Configuration of medical device
2-3. Measurement of vibration
2-4. Configuration of control device
3. Modified examples
3-1. First modified example
3-2. Second modified example
3-3. Third modified example
4. Hardware configuration example

### 5. Summary

### 1. Overview

In recent years, as a surgical operation system used when endoscopic surgery is performed, a master-slave type system (hereinafter, also referred to as a master-slave system) that enables an approach to an affected part without making a large incision in a patient's body has become known. In such a master-slave system, an operator (a user) such as a doctor operates a master device including an input interface, and a slave device including a medical instrument such as a forceps or tweezers is remotely operated in accordance with the operation of the master device by the operator. The slave device is configured as, for example, an arm device having a distal end for holding the medical device, and a position or posture of the medical device inside an abdominal cavity can be changed.

In such a master-slave system, in a case in which no contact state between the patient and the medical device is detected, the operator may be unaware that the medical device is in contact with the patient and damage the patient's tissue. Therefore, a method called hand-assisted laparoscopy surgery (HALS) in which an operator puts his or her hand through a hole made separately from a hole into which a medical device is inserted, and the operator performs an operation while directly touching tissues with his or her hand may be used. In this method, the operator can perform the operation while feeling a tactile sensation, but invasiveness in an abdominal cavity increases as compared with usual endoscopic surgery in which HALS is not used. Therefore, a technique has been developed in which a contact state between a patient and a medical device is detected on a slave device side, and feedback of the contact state is performed on a master device side to an operator. The technique includes, for example, a technique in which a sensor for measuring information on the contact state between the patient and the medical device is provided in the slave device, the information on the contact state measured by the sensor is transmitted to the master device, and a tactile sensation such as vibration is presented on the master device side to the operator.

However, in the above technique, in addition to the information regarding the contact state between the patient and the medical device, vibrations and driving sounds of a motor of the slave device, vibrations from an installation location, and vibrations unrelated to contacts such as noise from the surroundings (hereinafter also referred to as vibration noise) are cross-talked and measured by the sensor. Since such vibration noise is included in the vibrations presented to the operator as the tactile sensation by the master device, it may adversely affect the operation of the master device by the operator.

A master-slave system according to an embodiment of the present disclosure has been conceived in view of the above points and can reduce vibration noise transmitted to a sensor of a slave device. Hereinafter, each embodiment of the present disclosure having such an effect will be described in detail in order.

### 2. Embodiments of Present Disclosure

### 2-1. Configuration of Master-Slave System

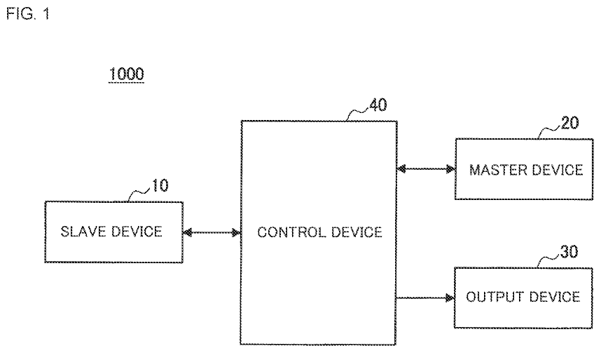

Hereinafter, a configuration of the master-slave system according to the embodiment of the present disclosure will be described with reference to FIGS. 1 to 3. FIG. 1 is an explanatory diagram showing a schematic configuration of the master-slave system according to the embodiment of the present disclosure. As shown in FIG. 1, a master-slave system 1000 according to an embodiment of the present disclosure is a master-slave type surgical system including a slave device 10, a master device 20, an output device 30, and a control device 40.

### (1) Slave Device 10

The slave device 10 is a device on a slave side in the master-slave system 1000. For example, the slave device 10 may be a robot (a robot having a link mechanism including active joints), which has one or two or more active joints and links connected to the active joints, for moving in response to an input operation to the master device 20. Also, the active joints are joints driven by a motor, an actuator, or the like.

Further, the slave device 10 includes, for example, motion sensors for measuring motions of the active joints at positions corresponding to each of the active joints. Examples of the motion sensors include an encoder and the like. Further, the slave device 10 includes, for example, drive mechanisms for driving the active joints at positions corresponding to each of the active joints. Examples of the drive mechanisms include a motor and a driver. Such drive mechanisms can be controlled by the control device 40, which will be described later.

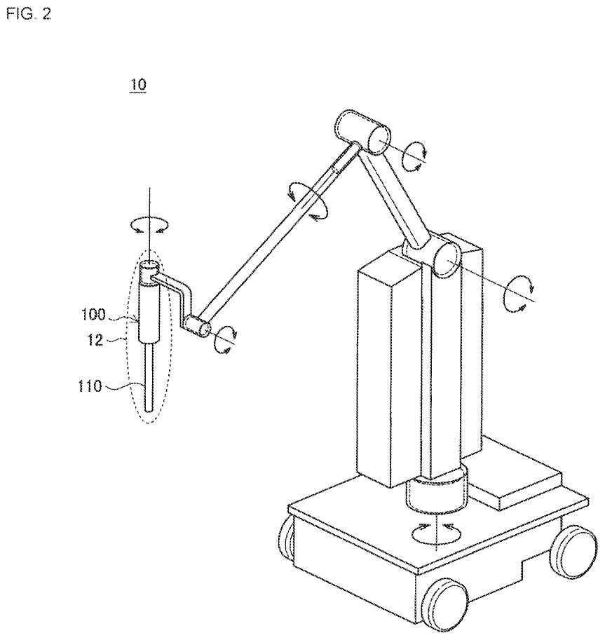

FIG. 2 is an explanatory diagram showing an example of the slave device according to the embodiment of the present disclosure. In the example shown in FIG. 2, the medical device 100 according to the present embodiment is provided at a distal end portion 12 that is a distal end portion of an arm of the slave device 10. A medical instrument 110 that comes into contact with a patient is further provided at a distal end portion of the medical device 100. A user remotely operates a position of the medical instrument 110 by operating the master device 20.

Also, the slave device 10 may have various sensors (for example, a force sensor, a vibration sensor, an origin sensor, a limit sensor, an encoder, a microphone, an acceleration sensor, etc.) in the medical device 100. For example, the slave device 10 has a vibration sensor in the medical device 100. The vibration sensor measures vibrations propagating in the medical instrument 110 when the medical instrument 110 comes into contact with the patient. Further, the slave device 10 has a microphone in the medical device 100. The microphone measures vibrations propagating in the air when the medical instrument 110 comes into contact with the patient. In addition, a place at which the above-mentioned various sensors are provided is not particularly limited, and the various sensors may be provided at any place of the medical device 100.

Also, the slave device 10 shown in FIG. 2 is an example, and the configuration of the slave device 10 according to the present embodiment is not limited to the example in FIG. 2.

### (2) Master Device 20

The master device 20 is a device on a master side in the master-slave system 1000. For example, the master device 20 may be a robot (a robot having a link mechanism including passive joints) which has one or two or more joints including a passive joint and links connected to the joints. Also, the passive joint is a joint that is not driven by a motor, an actuator, or the like.

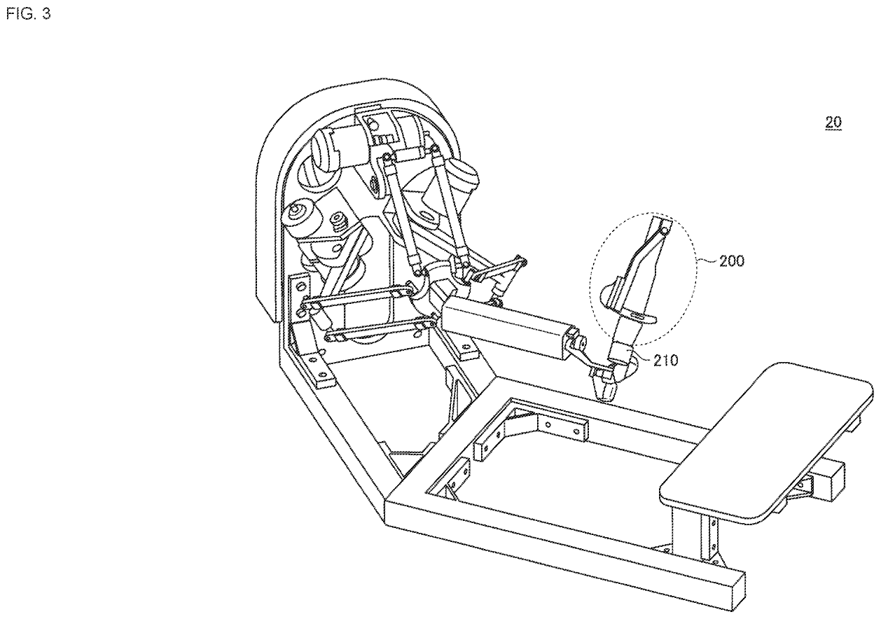

FIG. 3 is an explanatory diagram showing an example of the master device according to the embodiment of the present disclosure. In the example shown in FIG. 3, the master device 20 includes an operating body 200 provided on the link connected to the passive joint, and a force sensor 210 which measures a force applied to the operating body 200. Here, examples of the force sensor 210 according to the present embodiment include any sensor capable of measuring the force applied to the operating body 200, for example, “a force sensor of any type such as a type using a strain gauge”, “a tactile sensor of any type such as a type of obtaining a tactile sensation by measuring vibrations with a piezoelectric element or a microphone”, etc. Further, the master device 20 includes, for example, motion sensors for measuring motions of joints at positions corresponding to each of the joints.

In the present embodiment, the master device 20 is used to operate the slave device 10. For example, the master device 20 includes the operating body 200 that is an input interface of the master device 20. The user can move (remotely operate) the position of the medical instrument 110 of the slave device 10 described above by moving a position of the operating body 200.

Also, although FIG. 3 shows an example in which the operating body 200 provided in the master device 20 is a stylus type operating device, the operating body 200 according to the present embodiment is not limited to the example shown in FIG. 3. Examples of the operating body 200 according to the present embodiment include an operating device having an arbitrary shape, such as a glove-shaped operating device. Further, the operating body 200 according to the present embodiment may be any operating device that can be applied to a haptic device. In addition, the master device 20 may have a structure capable of replacing the operating body 200. Also, the configuration of the master device 20 according to the present embodiment is not limited to the example shown in FIG. 3 and may be any configuration.

Further, the operating body 200 has a vibration device for presenting vibrations generated when the patient comes into contact with the medical device 100 of the slave device 10 as a tactile sensation to the user. For the vibrating device, for example, a voice coil motor (VCM) type vibrating actuator is used. Also, a linear resonant actuator (LRA) or a piezoelectric element may be used for the vibrating device.

### (3) Output Device 30

The output device 30 outputs output information input from the control device 40, which will be described later. For example, the output device 30 may be a display device such as an installation type display or a head mounted display (HMD) mounted on the user's head. In a case in which image information is input as the output information from the control device 40, the output device 30 displays an image on the display device. Further, the output device 30 may be a sound output device such as a speaker or a headphone. In a case in which sound information is input as the output information from the control device 40, the output device 30 outputs sounds from the sound output device.

Also, the device used as the output device 30 is not limited to the above examples and any device may be used.

### (4) Control Device 40

The control device 40 controls each of the other devices included in the master-slave system 1000. For example, the control device 40 controls driving of the slave device 10. Specifically, the control device 40 receives information indicating an instruction for driving the arm of the slave device 10 from the master device 20 and controls the driving of the arm of the slave device 10 on the basis of the received information. The information indicating the instruction for driving the arm is input by the user operating the operating body 200 of the master device 20.

In addition, the control device 40 controls driving of the master device 20. Specifically, the control device 40 controls driving of the vibration device included in the operating body 200 of the master device 20 on the basis of information regarding the contact between the medical device 100 and the patient received from the slave device 10

Further, the control device 40 controls an output in the output device 30. Specifically, the control device 40 receives an image (a still image/a moving image) captured by a camera provided on the medical instrument 110 of the arm of the slave device 10 from the slave device 10 and transmits the image to the output device 30 to cause the output device 30 to output the image.

The control device 40 is connected to each of the other devices included in the master-slave system 1000 using an arbitrary communication method and transmits and receives information regarding control to each of the other devices via communication.

### 2-2. Configuration of Medical Device

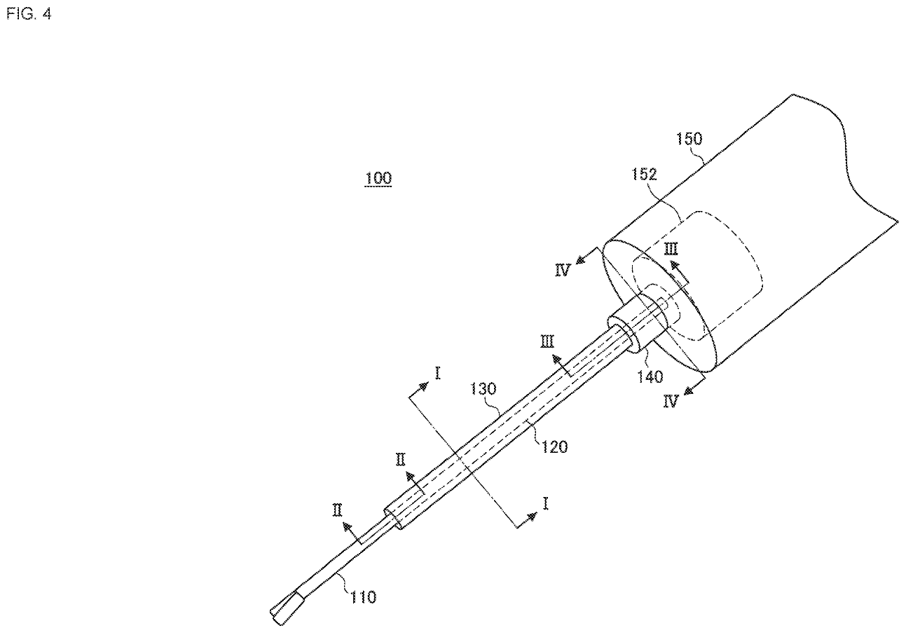

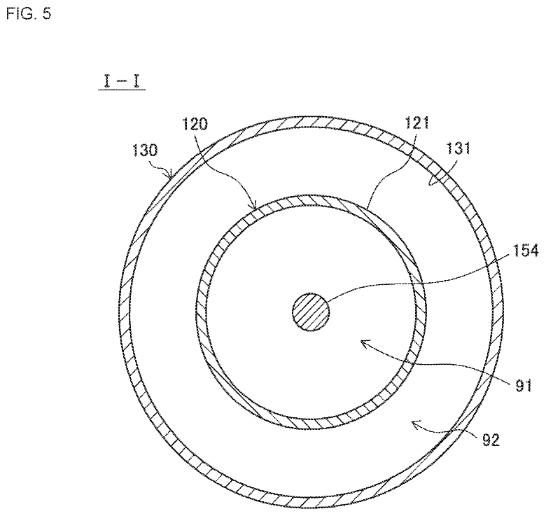

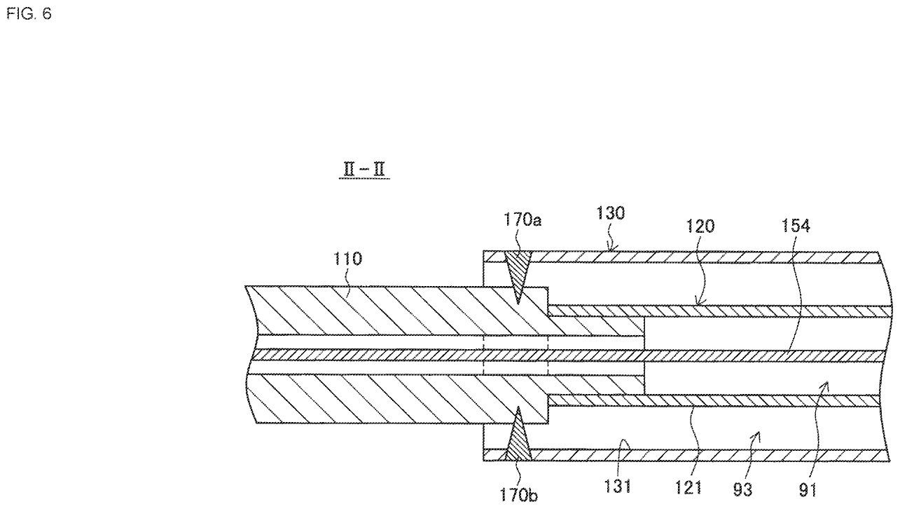

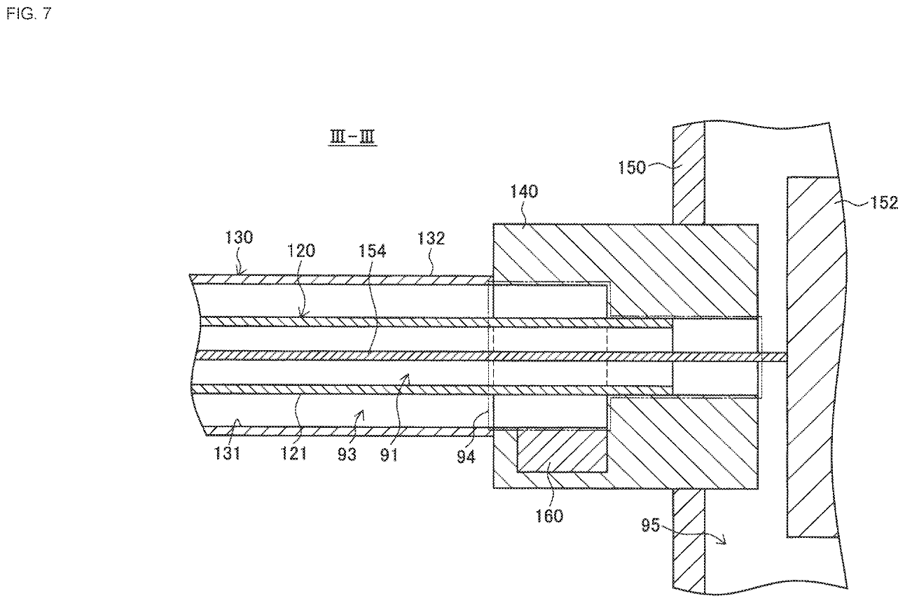

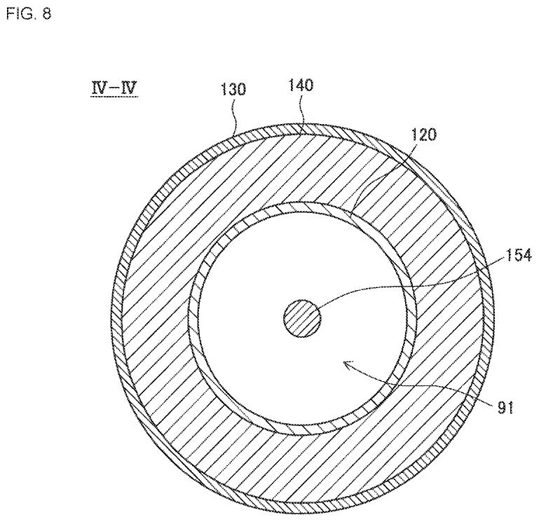

Hereinafter, a configurational example of the medical device according to the present embodiment will be described with reference to FIGS. 4 to 10. FIG. 4 is a perspective view showing an external configuration of the medical device according to the present embodiment. FIG. 5 is a cross-sectional view along line I-I in the medical device according to the present embodiment. FIG. 6 is a cross-sectional view along line II-II in the medical device according to the present embodiment. FIG. 7 is a cross-sectional view along line III-III in the medical device according to the present embodiment. FIG. 8 is a cross-sectional view along line IV-IV in the medical device according to the present embodiment.

As shown in FIG. 4, the medical device 100 is configured of the medical instrument 110, a pipe 120 (a first structural member), a pipe 130 (a second structural member), a coupling portion 140 (a third structural member), and a drive unit 150. In addition, as shown in FIG. 5, the pipe 120 is inserted into a hollow portion 92 of the pipe 130. Also, as shown in FIG. 6, the medical instrument 110 is coupled to the pipe 120. Further, as shown in FIG. 7, the drive unit 150 is coupled to the pipe 120 and the pipe 130 by the coupling portion 140.

Moreover, the medical device 100 has a power transmission mechanism which transmits power generated by driving the drive unit 150 to the medical instrument 110. The power transmission mechanism is configured to include, for example, a pulley sliding portion 152 and a wire 154. The pulley sliding portion 152 is provided inside the drive unit 150, for example, and is coupled to the drive unit 150 and the wire 154. The pulley sliding portion 152 has a function of transmitting the power of the drive unit 150 to the wire 154 as it is driven by driving the drive unit 150. The wire 154 is also coupled to the medical instrument 110 in addition to the pulley sliding portion 152. The wire 154 has a function of transmitting power of the pulley sliding portion 152 to the medical instrument 110 as it is driven by driving the pulley sliding portion 152. Then, the medical instrument 110 is driven by the power transmitted from the pulley sliding portion 152. Also, although only one wire 154 is shown in FIGS. 5 to 8 for convenience of explanation, a plurality of wires 154 may be provided depending on the configuration of the power transmission mechanism.

By having the above-described configuration, the medical device 100 can transmit the driving of the drive unit 150 to the medical instrument 110. Also, the power transmission mechanism may be realized by a link mechanism.

The medical instrument 110 is, for example, a forceps type instrument. The medical instrument 110 is connected to the drive unit 150 via the power transmission mechanism and is driven by the drive unit 150. The pipe 120 is a structural member that has a hollow portion 91 and has opening portions at both ends thereof. A surface of the pipe 120 is also hereinafter referred to as an outer wall 121 of the pipe 120. The pipe 120 is, for example, a structural member of which a shape of a cross-section is circular. Also, the shape of the cross-section of the pipe 120 is not limited to a circular shape and may be any shape. In addition, a length of the pipe 120 is not particularly limited and may be any length. Further, a shape of the pipe 120 is not particularly limited and may be any shape. For example, the shape of the pipe 120 may be a linear shape or a curved shape.

The pipe 130 is a structural member that has a hollow portion 92 and has opening portions at both ends thereof. A surface of the hollow portion 92 of the pipe 130 is also hereinafter referred to as an inner wall 131 of the pipe 130. The pipe 130 is, for example, a structural member of which a shape of a cross-section is circular. Also, the shape of the cross-section of the pipe 130 is not limited to a circular shape and may be any shape. In addition, a length of the pipe 130 is not particularly limited and may be any length, but the length is preferably equivalent to that of the pipe 120. Further, a shape of the pipe 120 is not particularly limited and may be any shape. For example, the shape of the pipe 120 may be a linear shape or a curved shape. The coupling portion 140 has a hollow portion 94 therein and is a structural member that couples the pipe 120, the pipe 130, and the drive unit 150.

For example, the medical instrument 110 is provided in one opening portion of the pipe 120. Specifically, as shown in FIG. 6, the medical instrument 110 is inserted into the one opening portion of the pipe 120 and fixed by fastening members 170 from an outside of the pipe 130. Also, the number of the fastening members 170 used for fixing the medical instrument 110 is not particularly limited and may be any number. For example, as shown in FIG. 6, the pipe 120 is fixed by two fastening members 170, a fastening member 170a and a fastening member 170b. Further, the pipe 120 does not necessarily have to be fixed by the fastening members 170.

Also, for example, the coupling portion 140 is provided in the other opening portion of the pipe 120. Specifically, as shown in FIG. 7, the pipe 120 is inserted into the hollow portion 94 of the coupling portion 140. Further, the drive unit 150 is coupled to a side opposite to a side of the coupling portion 140 to which the pipe 120 is coupled. That is, the medical instrument 110 is positioned on the one opening portion side of the pipe 120, and the drive unit 150 is positioned on the other opening portion side of the pipe 120. Also, the pipe 120 may be inserted through the hollow portion 94 of the coupling portion 140 as described above and may be coupled to the coupling portion 140 by fixing an end portion of the pipe 120 and the coupling portion 140 in close contact with each other.

Further, the power transmission mechanism is inserted through the hollow portion 91 of the pipe 120. As shown in FIGS. 6 and 7, for example, the wire 154 coupled to the pulley sliding portion 152 is inserted into the hollow portion 91 of the pipe 120. In this case, the wire 154 couples the medical instrument 110 and the drive unit 150 such that the medical instrument 110 is positioned on the one opening portion side of the pipe 120 and the drive unit 150 is positioned on the other opening portion side of the pipe 120. In addition, the hollow portion 91 of the pipe 120 is connected to a hollow portion 95 of the drive unit 150. Hereinafter, the one opening portion of the pipe 120 is also referred to as an opening portion of the pipe 120 on the medical instrument 110 side. Also, the other opening portion of the pipe 120 is also referred to below as an opening portion of the pipe 120 on the drive unit 150 side.

For example, as shown in FIGS. 4 to 7, the pipe 120 is inserted into the hollow portion 92 of the pipe 130. In this case, the pipe 120 is inserted into the hollow portion 92 of the pipe 130 such that the medical instrument 110 is positioned on one opening portion side of the pipe 130 and the drive unit 150 is positioned on the other opening portion side of the pipe 130. Hereinafter, the one opening portion of the pipe 130 is also referred to as an opening portion of the pipe 130 on the medical instrument 110 side. Further, the other opening portion of the pipe 130 is also referred to below as an opening portion of the pipe 130 on the drive unit 150 side.

Since the pipe 120 is inserted into the hollow portion 92 of the pipe 130, a size of a diameter of the cross-section of the pipe 130 is preferably larger than a size of a diameter of the cross-section of the pipe 120. Further, the pipe 120 is preferably inserted into the hollow portion 92 of the pipe 130 with a spatial margin between the outer wall 121 of the pipe 120 and the inner wall 131 of the pipe 130. In addition, the power transmission mechanism is inserted through the pipe 120. For that reason, it is preferable that the size of the diameter of the cross-section of the pipe 120 be smaller than the size of the diameter of the cross-section of the pipe 130 and within a range equal to or larger than a size that allows the power transmission mechanism to be inserted into the hollow portion 91. Also, as long as it is within this range, the size of the diameter of the cross-section of the pipe 120 is not particularly limited.

As described above, the fastening members 170 that fix the medical instrument 110 are provided in the vicinity of the opening portion of the pipe 130 on the medical instrument 110 side. On the other hand, for example, the coupling portion 140 is provided in the opening portion of the pipe 130 on the drive unit 150 side. Specifically, as shown in FIG. 7, the pipe 130 is coupled to the coupling portion 140. Also, a method of coupling the pipe 130 to the coupling portion 140 is not particularly limited. For example, the pipe 130 may be coupled to the coupling portion 140 by being inserted into the hollow portion 94 of the coupling portion 140, similarly to the pipe 120.

The coupling portion 140 couples the outer wall 121 of the pipe 120 and the inner wall 131 of the pipe 130. For example, referring to FIG. 7, the coupling portion 140 is coupled to the pipe 120 and the pipe 130 to close the opening portion on the drive unit 150 side between the pipe 120 and the pipe 130. Specifically, as shown in FIG. 8, the coupling portion 140 is provided such that a drive unit 150 side of a space 93 between the outer wall 121 of the pipe 120, the inner wall 131 of the pipe 130, and the coupling portion 140 is closed and the hollow portion 91 of the pipe 120 through which the wire 154 passes is not closed.

As described above, the medical device 100 has the configuration in which the pipe 120 is inserted into the hollow portion 92 of the pipe 130 and the opening portion between the pipe 120 and the pipe 130 on the drive unit 150 side is closed by the coupling portion 140. Thus, the medical device 100 can separate the hollow portion 91 of the pipe 120 and the hollow portion 95 of the drive unit 150 on the drive unit 150 side from the space 93 between the outer wall 121 of the pipe 120 and the inner wall 131 of the pipe 130. Hereinafter, separating the hollow portion 91 and the hollow portion 95 from the space 93 in this way is also referred to as space separation.

The medical device 100 further has a sensor unit 160 that measures vibrations of the space 93 on the medical instrument 110 side between the outer wall 121 of the pipe 120, the inner wall 131 of the pipe 130, and the coupling portion 140. There are at least two types of vibrations measured by the sensor unit 160. A first vibration is, for example, a vibration propagating in the air (hereinafter, also referred to as air vibration or sound). A second vibration is, for example, a vibration propagating in the medical device 100 (hereinafter, also referred to as housing vibration). In addition, there are at least three types of factors that cause the vibrations according to the present embodiment. A first factor is, for example, the contact between the medical instrument 110 and the patient. A second factor is, for example, the driving of the drive unit 150. A third factor is, for example, an external factor. The two types of vibrations mentioned above are included in each of the vibrations generated by each of the three types of factors. That is, the vibrations according to the present embodiment include at least six types of vibrations.

More specifically, the first and second vibrations are sound generated by the contact between the medical instrument 110 and the patient (hereinafter, also referred to as contact sound) and housing vibration (hereinafter, also referred to as contact vibration). Third and fourth vibrations are sound generated by driving the drive unit 150 (hereinafter, also referred to as driving sound) and housing vibration (hereinafter, also referred to as driving vibration). Fifth and sixth vibrations are sound generated by an external factor (hereinafter, also referred to as external sound) and housing vibration (hereinafter, also referred to as external vibration). Also, in the present embodiment, the sensor unit 160 preferably measures at least the contact sound and also the contact vibration. The vibrations other than the contact sound and the contact vibration become causes of noise. For that reason, the sensor unit 160 preferably does not measure the vibrations other than the contact sound and the contact vibration. The vibrations other than the contact sound and the contact vibration are also hereinafter referred to as vibration noise.

In the present embodiment, the sensor unit 160 preferably measures only the contact sound, but also measures the vibrations other than the contact sound depending on an installation location of the sensor unit 160. Therefore, the sensor unit 160 is preferably provided at a position at which the vibrations other than the contact sound are further reduced when measured. For example, the sensor unit 160 according to the present embodiment is provided at the position shown in FIG. 7. The position of the sensor unit 160 shown in FIG. 7 is a position on the drive unit 150 side of the space 93 between the outer wall 121 of the pipe 120, the inner wall 131 of the pipe 130, and the coupling portion 140 on the medical instrument 110 side. First, when the sensor unit 160 is provided in the space 93, it becomes difficult or impossible to measure the driving sound propagating in the air of the hollow portion 91 of the pipe 120. Therefore, the sensor unit 160 can reduce the measured vibration noise. Further, the sensor unit 160 is installed at a proximal end that is on the drive unit 150 side rather than a distal end that is on the medical instrument 110 side of the medical device 100, so that the medical instrument 110 can be kept cleaner.

Also, the sensor unit 160 is provided in the pipe 130 or the coupling portion 140. In the example shown in FIG. 7, the sensor unit 160 is provided in the coupling portion 140. Further, the sensor unit 160 shown in FIG. 7 is provided at a position of the hollow portion 94 of the coupling portion 140 in the space 93 between the outer wall 121 of the pipe 120, the inner wall 131 of the pipe 130, and the coupling portion 140 on the medical instrument 110 side. The sensor unit 160 may be provided in the pipe 130, but can be provided closer to the proximal end side by being provided in the coupling portion 140 that is closer to the drive unit 150 side than the pipe 130.

Further, a sensor unit different from the sensor unit 160 provided in the space 93 may be provided on an outside of the medical device 100 such as an outer wall 132 of the pipe 130. Since the different sensor unit is located outside the medical device 100, it can detect the external sound and the external vibration that are vibration noise. In a case in which the vibrations measured by the sensor unit 160 include at least one of the external sound and the external vibration, the medical device 100 can remove vibration noise from the vibrations measured by the sensor unit 160 on the basis of the external sound or the external vibration measured by the different vibration unit.

In addition, the drive unit 150 has the motor (not shown) inside or outside the hollow portion 95. In a case in which the motor is inside the hollow portion 95 of the drive unit 150, for example, driving sounds of the motor propagate in the hollow portion 91 and the hollow portion 95, but do not propagate in the space 93 due to the space separation, and are not measured by the sensor unit 160. On the other hand, in a case in which the motor is outside the hollow portion 95 of the drive unit 150, the drive sounds of the motor may go around through the opening portion on the medical instrument 110 side between the pipe 120 and the pipe 130 to propagate in the space 93. However, the driving sounds of the motor go around the opening portion on the medical instrument 110 side between the pipe 120 and the pipe 130 and are attenuated by the time they are transmitted to the sensor unit 160. Therefore, the driving sounds of the motor are unlikely to be measured by the sensor unit 160.

By having the spatially separated configuration described above, the medical device 100 can reduce the vibration noise transmitted to the sensor unit 160. As a result, the medical device 100 can also improve a signal-noise (SN) ratio (a ratio of signals to noise) without using special signal processing.

Further, materials that reduce vibrations propagating in the drive unit 150 may be used for components constituting the drive unit 150 that is a noise source. Examples of the materials for reducing the vibrations propagating in the drive unit 150 include carbon and a foaming agent having a porous structure. By using these materials for the components constituting the drive unit 150, the sensor unit 160 can further reduce the measured vibration noise.

### 2-3. Measurement of Vibration

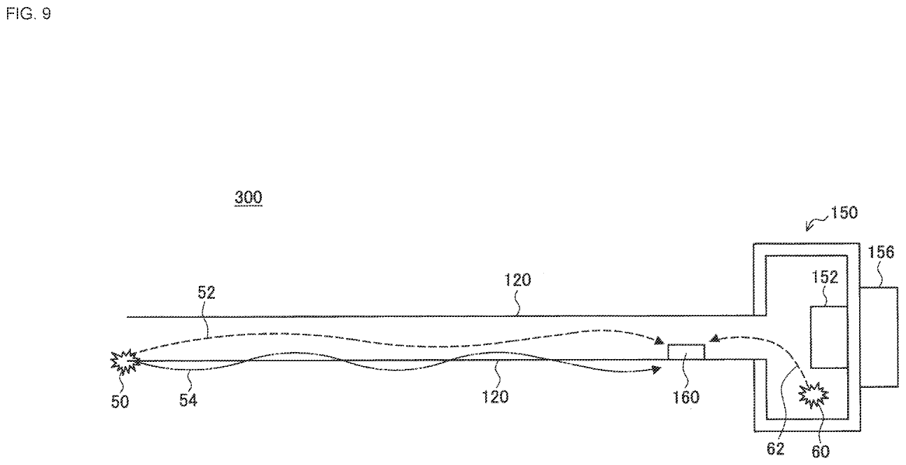

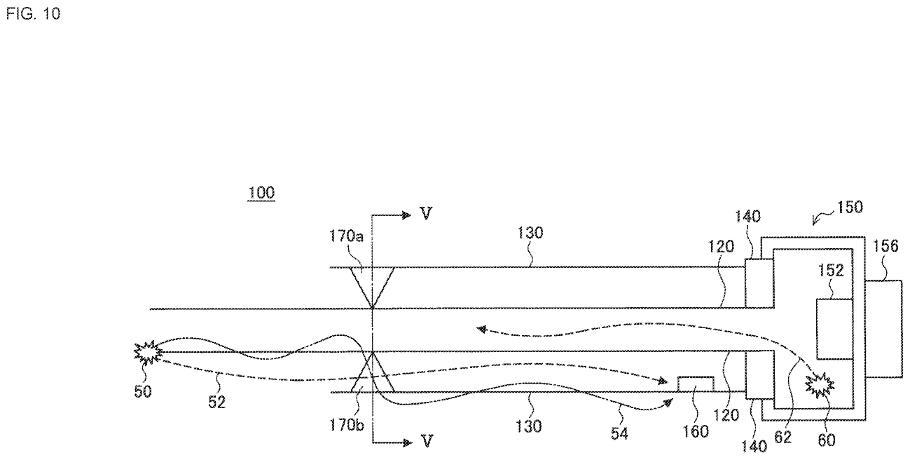

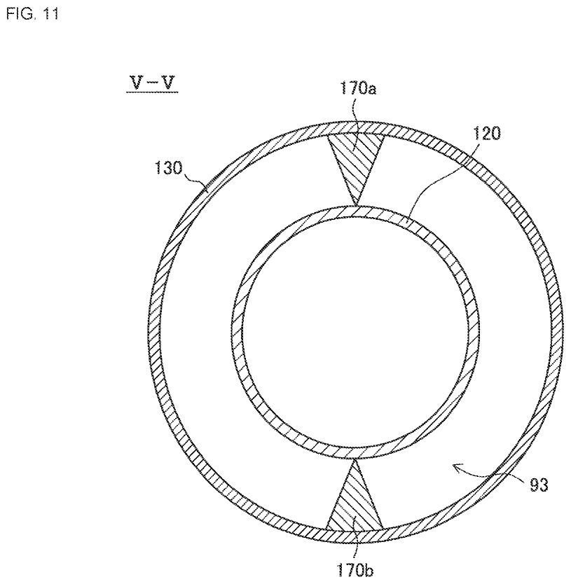

Hereinafter, an example of measurement of vibration according to the present embodiment will be described with reference to FIGS. 9 to 11. FIG. 9 is a simplified diagram showing a medical device according to a comparative example. FIG. 10 is a simplified diagram showing the medical device according to the present embodiment. FIG. 11 is a cross-sectional view along line V-V in the medical device according to the present embodiment. Also, in the simplified diagram of the medical device, the description of the medical instrument 110 and the wire 154 provided in the one opening portion of the pipe 120 will be omitted for convenience of explanation. Further, it is assumed that the motor 156 is provided on the outside of the drive unit 150 and the motor 156 is coupled to the pulley sliding portion 152 provided inside the drive unit 150.

The medical device 300 according to the comparative example shown in FIG. 9 is different from the medical device 100 according to the present embodiment in that the pipe 130 is not provided and the sensor unit 160 is provided in the pipe 120. Thus, vibration 52 (the contact sound) that is generated at a position 50 due to a contact between the medical instrument 110 and a target object and propagates in the air in the hollow portion 91 of the pipe 120 and vibration 54 (the contact vibration) propagating in the pipe 120 are measured by the sensor unit 160. Further, vibration 62 (the driving sound) generated at a position 60 by driving the drive unit 150 is also measured by the sensor unit 160. For that reason, the medical device 300 cannot reduce the vibration noise transmitted to the sensor unit 160. This is because the medical device 300 according to the comparative example is not spatially separated. Specifically, this is because the medical device 300 does not include the pipe 130 and the coupling portion 140 and thus the air in which the vibration 52 (contact sound) and the vibration 62 (driving sound) propagate is not separated. In addition, this is also because the sensor unit 160 is provided at a position at which both of the vibration 52 (contact sound) and the vibration 62 (driving sound) are measured.

On the other hand, in the medical device 100 according to the present embodiment shown in FIG. 10, the pipe 130 is provided outside the pipe 120, and the sensor unit 160 is provided in the pipe 130. Also, the opening portion of the pipe 130 between the outer wall of the pipe 120 and the outer wall of the pipe 130 on the drive unit 150 side is closed by the coupling portion 140. Thus, the vibration 52 (contact sound) that is generated at the position 50 due to a contact between the medical instrument 110 and a target object and propagates in the air in the space 93 between the outer wall 121 of the pipe 120, the inner wall 131 of the pipe 130, and the coupling portion 140 on the medical instrument 110 side is measured by the sensor unit 160. Further, the vibration 54 (contact vibration) propagating in the pipe 120, the fastening members 170, and the pipe 130 is also measured by the sensor unit 160. On the other hand, since the vibration 62 (driving sound) generated at the position 60 by driving the drive unit 150 propagates in the air in the pipe 120 in which the sensor unit 160 is not provided, it is difficult to be or not measured by the sensor unit 160. This is because the medical device 100 is spatially separated. In addition, in a case in which the motor is provided outside the hollow portion 95, the driving sounds of the motor go around toward the medical instrument 110 side, enters into the space 93 through a gap on the medical instrument 110 side, and may be measured by the sensor unit 160 as the vibration noise. However, the driving sounds of the motor are attenuated by the time they are measured by the sensor unit 160 by going around toward the medical instrument 110 side. Therefore, the medical device 100 can reduce the vibration noise transmitted to the sensor unit 160.

Further, as shown in the cross-sectional view of the positions of the fastening members 170 shown in FIG. 11, the space 93 is not closed between the fastening member 170a and the fastening member 170b. For that reason, the vibration 52 can propagate in the air in the space 93.

As described above, the medical device 100 according to the present embodiment can achieve the above-mentioned effects peculiar to the present application by providing the medical device component configured of the pipe 130, the coupling portion 140, and the sensor unit 160 having the above-mentioned relationship with other components of the medical device 100.

### 2-4. Functional Configuration of Control Device

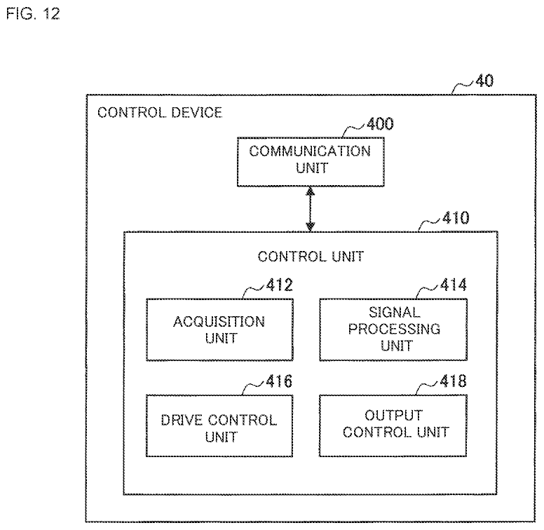

Hereinafter, an example of a functional configuration of the control device 40 according to the embodiment of the present disclosure will be described with reference to FIG. 12. FIG. 12 is a block diagram showing a functional configuration example of the control device according to the embodiment of the present disclosure. As shown in FIG. 12, the control device 40 includes a communication unit 400 and a control unit 410.

### (1) Communication Unit 400

The communication unit 400 has a function of communicating with other devices. For example, the communication unit 400 outputs information received from other devices to the control unit 410 in communication with other devices. Specifically, the communication unit 400 outputs sensing information received from the slave device 10 to the control unit 410. Further, the communication unit 400 outputs control information received from the master device 20 to the control unit 410.

The communication unit 400 transmits information input from the control unit 410 to other devices in communication with other devices. Specifically, the communication unit 400 transmits information regarding driving processing input from the control unit 410 to the slave device 10. In addition, the communication unit 400 transmits information regarding presentation of detection information input from the control unit 410 to the master device 20. Further, the communication unit 400 transmits information regarding output processing input from the control unit 410 to the output device 30.

### (2) Control Unit 410

The control unit 410 has a function of controlling the entire operations of the master-slave system 1000. In order to realize this function, as shown in FIG. 12, the control unit 410 according to the present embodiment includes an acquisition unit 412, a signal processing unit 414, a drive control unit 416, and an output control unit 418.

### (2-1) Acquisition Unit 412

The acquisition unit 412 has a function of acquiring sensing information. For example, the acquisition unit 412 acquires the sensing information measured by the sensor unit 160 included in the medical device 100 of the slave device 10 as signals via the communication unit 400. In addition, the acquisition unit 412 outputs signals related to the acquired sensing information to the signal processing unit 414.

Further, the acquisition unit 412 acquires the sensing information measured by the force sensor 210 included in the master device 20 as signals via the communication unit 400. In addition, the acquisition unit 412 outputs signals related to the acquired sensing information to the drive control unit 416.

### (2-2) Signal Processing Unit 414

The signal processing unit 414 has a function of processing signals related to operations of the slave device 10 and the master device 20 on the basis of the signals input from the acquisition unit 412. For example, the signal processing unit 414 generates signals related to driving of the arm of the slave device 10 on the basis of signals of information related to the user's input operation measured by the force sensor 210 of the master device 20. In addition, the signal processing unit 414 outputs the generated signals to the drive control unit 416.

Also, the signal processing unit 414 generates signals related to driving of the vibration device of the master device 20 on the basis of signals of information on the contact between the medical device 100 and the patient measured by the sensor unit 160 of the slave device 10. Specifically, in a case in which only signals of the information measured by the sensor unit 160 are input, the signal processing unit 414 generates signals based on the signals of the information measured by the sensor unit 160. In addition, the signal processing unit 414 outputs the generated signals to the drive control unit 416.

### (2-3) Drive Control Unit 416

The drive control unit 416 has a function of controlling driving of the slave device 10 and the master device 20 on the basis of signals input from the signal processing unit 414. For example, the drive control unit 416 receives signals related to the information measured by the force sensor 210 of the master device 20 from the signal processing unit 414 and controls driving of the arm of the slave device 10 on the basis of the received information. Further, the drive control unit 416 receives signals related to the information measured by the sensor unit 160 of the slave device 10 from the signal processing unit 414 and controls driving of the vibration device of the master device 20 on the basis of the received information.

### (2-4) Output Control Unit 418

The output control unit 418 controls information output to the output device 30. For example, the output control unit 418 receives an image (a still image/a moving image) captured by a camera provided on the medical instrument 110 of the arm of the slave device 10 via the communication unit 400 and transmits the received image to the output device 30 to cause the output device 30 to output the image.

### 3. Modified Examples

Hereinafter, modified examples of the embodiment of the present disclosure will be described. Also, the modified examples described below may be applied alone to the embodiment of the present disclosure or may be applied in combination to the embodiment of the present disclosure. Further, the modified examples may be applied in place of the configurations described in the embodiment of the present disclosure or may be additionally applied to the configurations described in the embodiment of the present disclosure.

### 3-1. First Modified Example

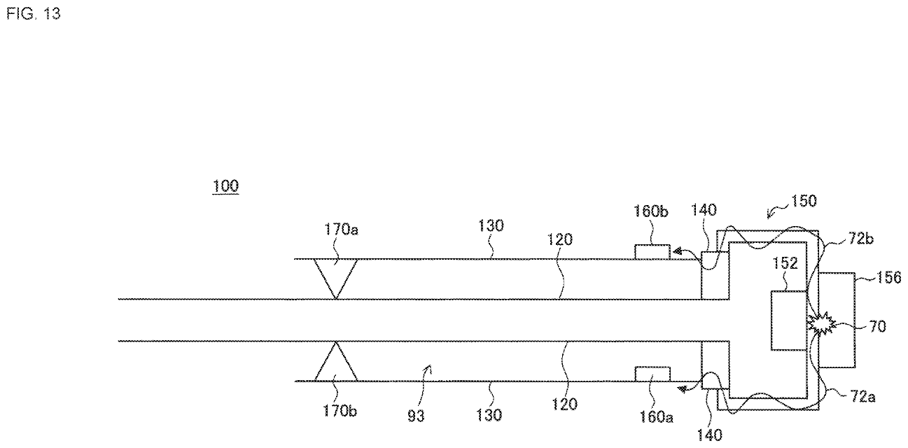

Hereinafter, a first modified example according to the embodiment of the present disclosure will be described with reference to FIG. 13. FIG. 13 is an explanatory diagram showing the first modified example according to the embodiment of the present disclosure.

In the above-described embodiment, an example in which the medical device 100 has only one sensor unit 160 has been described. In the first modified example, an example in which the medical device 100 further includes a sensor unit different from the sensor unit 160 will be described.

For example, as shown in FIG. 13, the medical device 100 has a sensor unit 160a and a sensor unit 160b. The sensor unit 160a is provided at the same position as the sensor unit 160 in the above-described embodiment, and the sensor unit 160b is provided at a position different from that of the sensor unit 160a. In addition, the vibration measured by the sensor unit 160b is used to remove vibration noise included in the vibration measured by the sensor unit 160a. For that reason, the sensor unit 160b is preferably provided at a position at which the vibration noise desired to be removed from the vibration measured by the sensor unit 160a can be measured. For example, in the example shown in FIG. 13, vibration 72a generated at a position 70 by driving the motor 156 propagates in a housing of the medical device 100 and is transmitted to the sensor unit 160a. Thus, the vibration measured by the sensor unit 160a includes driving vibration that becomes vibration noise. Therefore, in a case in which it is desired to remove the driving vibration, the sensor unit 160b may be provided at a position at which the driving vibration can be measured. For example, as shown in FIG. 13, the sensor unit 160b may be provided to be in contact with the outer wall side of the pipe 130 and not to come into contact with the air on the inner wall 131 side. As a result, since the sensor unit 160b does not measure the vibration related to the space 93, it is possible to measure the vibration closer to the vibration noise included in the vibration measured by the sensor unit 160a.

Also, the sensor unit 160b may be provided on the same structural member as the structural member on which the sensor unit 160a is provided. For example, in the example shown in FIG. 13, the sensor unit 160a is provided on the pipe 130. The vibration 72a is attenuated by being transmitted through the pipe 130 before being measured by the sensor unit 160a. Therefore, vibration 72b is also transmitted through the pipe 130 and then measured by the sensor unit 160b, and thus the sensor unit 160b can measure vibration closer to the vibration noise included in the vibration measured by the sensor unit 160a. Further, each sensor unit may be provided at a position at which the vibrations 72a and 72b are transmitted through the same structural member and transmission distance from the position 70 to each sensor unit. Thus, the sensor unit 160b can measure the vibration closer to the vibration noise included in the vibration measured by the sensor unit 160a. In addition, by removing the vibration noise measured by the sensor unit 160b from the vibration measured by the sensor unit 160a, the medical device 100 can further reduce the vibration noise.

Also, removing the vibration noise are performed by the signal processing unit 414. For example, in a case in which signals of information related to vibration noise measured by another sensor unit 160b are input in addition to signals of information measured by the sensor unit 160a, the signal processing unit 414 performs processing of removing signals of the information related to the vibration noise from signals of the information measured by the sensor unit 160a. In addition, the signal processing unit 414 outputs the processed signals to the drive control unit 416.

### 3-2. Second Modified Example

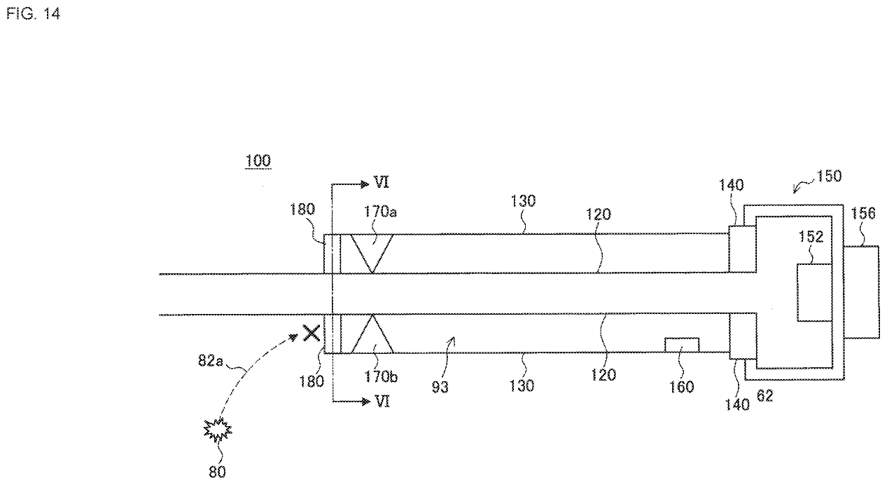

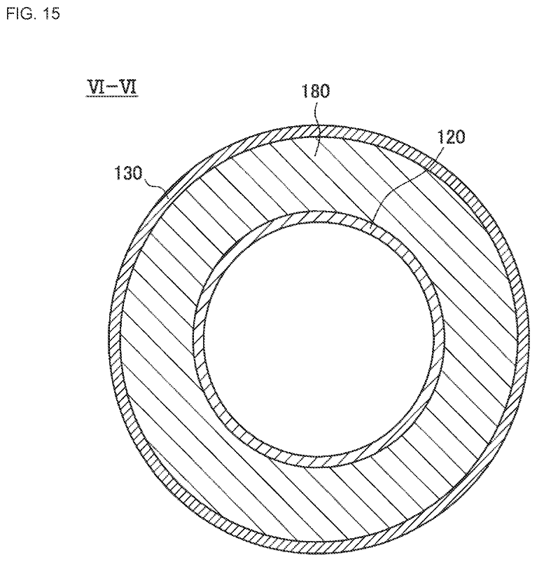

Hereinafter, a second modified example according to the embodiment of the present disclosure will be described with reference to FIGS. 14 and 15. FIG. 14 is an explanatory diagram showing the second modified example according to the embodiment of the present disclosure. FIG. 15 is a cross-sectional view along line VI-VI in the medical device according to the embodiment of the present disclosure.

In the above-described embodiment, an example in which the opening portion between the pipe 120 and the pipe 130 of the space 93 on the medical instrument 110 side is not closed has been described. In the second modified example, an example of closing the opening portions will be described.

For example, the medical device 100 further includes an O-ring 180 (a fourth structural member). As shown in FIG. 14, the O-ring 180 couples the pipe 120 and the pipe 130 on a side closer to the medical instrument 110 than the sensor unit 160 of the space 93. By coupling the pipe 120 and the pipe 130 using the O-ring 180, as shown in FIG. 15, the opening portion between the pipe 120 and the pipe 130 on the medical instrument 110 side is closed. Thus, the space 93 and a space in which vibration 82a that is the external sound propagates are spatially separated, and thus the vibration 82a is not transmitted to the sensor unit 160a. Therefore, the medical device 100 can reduce the vibration noise measured by the sensor unit 160a. Further, the medical device 100 can reduce the vibration noise by performing the spatial separation using the O-ring 180 without performing special signal processing.

### 3-3. Third Modified Example

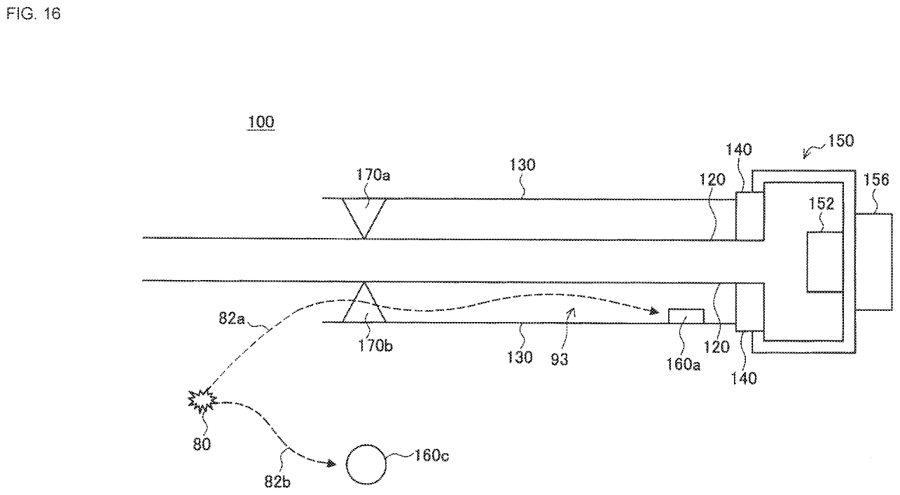

Hereinafter, a third modified example according to the embodiment of the present disclosure will be described with reference to FIG. 16. FIG. 16 is an explanatory diagram showing the third modified example according to the embodiment of the present disclosure.

In the above-described first modified example, an example in which the medical device 100 further includes the sensor unit different from the sensor unit 160 has been described. In the third modified example, an example in which a sensor unit different from the sensor unit 160 is provided outside the medical device 100 will be described.

For example, a sensor unit 160c different from the sensor unit 160a is further provided outside the medical device 100, and the sensor unit 160c measures vibration generated outside the medical device 100. The vibration generated outside the medical device 100 is, for example, at least one of the external sound and the external vibration. Specifically, as shown in FIG. 16, it is assumed that vibrations 82a and 82b (for example, external sounds) are generated at a position 80 due to external factors. The vibration 82a propagates in the space 93 from between the fastening members 170a and 170b and is measured by the sensor unit 160a. On the other hand, the vibration 82b propagates in the air outside the medical device 100 and is measured by the sensor unit 160c provided outside the medical device 100. In addition, the medical device 100 can further reduce the vibration noise by removing the vibration noise measured by the sensor unit 160c from the vibration measured by the sensor unit 160a. Further, processing of removing the vibration noise is performed by the signal processing unit 414 as in the first modified example.

### 4. Hardware Configuration Example

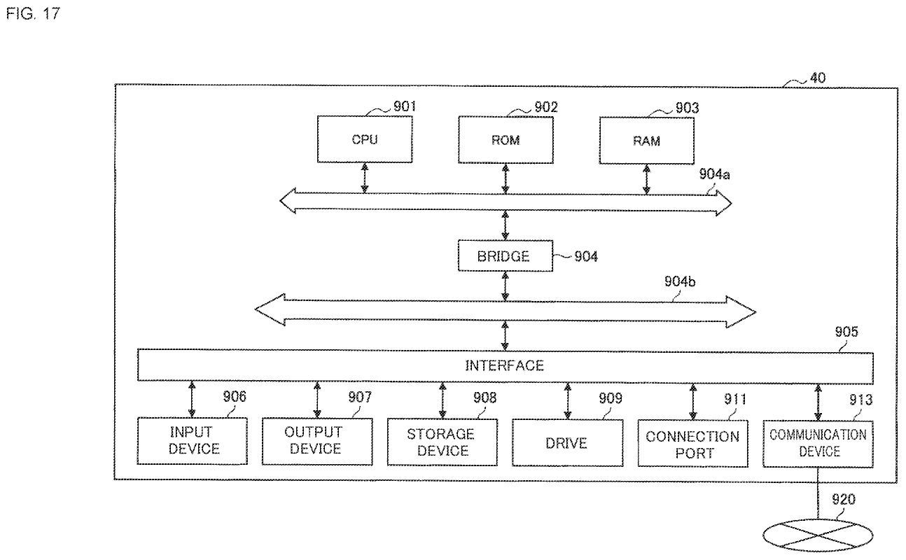

As described above, the embodiment of the present disclosure has been described. Finally, a hardware configuration according to the embodiment of the present disclosure will be described with reference to FIG. 17. FIG. 17 is a block diagram showing an example of the hardware configuration of the control device according to the embodiment of the present disclosure. Also, the control device 40 shown in FIG. 17 can realize the function of the control device 40 shown in FIG. 12. Information processing performed by the control device 40 according to the present embodiment is realized by the cooperation between software and hardware described below.

As shown in FIG. 17, the control device 40 includes a central processing unit (CPU) 901, a read only memory (ROM) 902, and a random access memory (RAM) 903. Also, the control device 40 includes a host bus 904a, a bridge 904, an external bus 904b, an interface 905, an input device 906, an output device 907, a storage device 908, a drive 909, a connection port 911, and a communication device 913. Further, the hardware configuration shown here is an example, and some of the components may be omitted. In addition, the hardware configuration may further include components other than the components shown here.

### (CPU 901, ROM 902, and RAM 903)

The CPU 901 functions as, for example, an arithmetic processing unit or a control device and controls all or some of operations of each component on the basis of various programs recorded in the ROM 902, the RAM 903, or the storage device 908. The ROM 902 is a means for storing a program read into the CPU 901, data used for calculation, and the like. In the RAM 903, for example, a program read into the CPU 901, various parameters that change as appropriate when the program is executed, and the like are temporarily or permanently stored. These are connected to each other by the host bus 904a configured of a CPU bus or the like. The CPU 901, the ROM 902, and the RAM 903 can realize the function of the control unit 410 described with reference to FIG. 12, for example, in cooperation with software.

### (Host Bus 904a, Bridge 904, External Bus 904b, and Interface 905)

The CPU 901, the ROM 902, and the RAM 903 are connected to each other via, for example, the host bus 904a capable of high-speed data transmission. On the other hand, the host bus 904a is connected to the external bus 904b, which has a relatively low data transmission speed, via, for example, the bridge 904. Further, the external bus 904b is connected to various components via the interface 905.

### (Input Device 906)

The input device 906 is realized by a device in which information is input by a user, such as a mouse, a keyboard, a touch panel, a button, a microphone, a switch, and a lever. Also, the input device 906 may be, for example, a remote control device using infrared rays or other radio waves, or an externally connected device such as a mobile phone or a PDA that supports the operation of the control device 40. Further, the input device 906 may include, for example, an input control circuit that generates an input signal on the basis of information input by the user using the above input means and outputs the input signal to the CPU 901. By operating the input device 906, the user of the control device 40 can input various data to the control device 40 and instruct processing operations.

Alternatively, the input device 906 may be formed by a device that detects information related to the user. For example, the input device 906 may include various sensors such as an image sensor (for example, a camera), a depth sensor (for example, a stereo camera), an acceleration sensor, a gyro sensor, a geomagnetic sensor, an optical sensor, a sound sensor, a ranging sensor (for example, a time of flight (ToF) sensor), and a force sensor. Also, the input device 906 may acquire information about a state of the control device 40 itself, such as a posture and a moving speed of the control device 40, or information about surrounding environment of the control device 40, such as brightness and noise around the control device 40. Further, the input device 906 may also include a global navigation satellite system (GNSS) module that receives GNSS signals from GNSS satellites (for example, global positioning system (GPS) signals from GPS satellites) and measures position information including the latitude, longitude and altitude of the device. Also, regarding the position information, the input device 906 may detect the position through Wi-Fi (registered trademark), transmission and reception of signals to and from mobile phones, PHSs or smartphones, or short-range communication.

### (Output Device 907)

The output device 907 is formed by a device capable of visually or audibly notifying the user of the acquired information. Such a device includes a display device such as a CRT display device, a liquid crystal display device, a plasma display device, an EL display device, a laser projector, an LED projector and a lamp, an sound output device such as a speaker and a headphone, and a printer device. The output device 907 outputs, for example, results obtained by various processes performed by the control device 40. Specifically, the display device visually displays the results obtained by various processes performed by the control device 40 in various formats such as texts, images, tables, and graphs. On the other hand, the sound output device converts an sound signal composed of reproduced sound data, acoustic data, etc., into an analog signal and outputs it audibly.

### (Storage Device 908)

The storage device 908 is a data storage device formed as an example of a storage unit of the control device 40. The storage device 908 is realized by, for example, a magnetic storage unit device such as an HDD, a semiconductor storage device, an optical storage device, an optical magnetic storage device, or the like. The storage device 908 may include a storage medium, a recording device that records data on the storage medium, a reading device that reads data from the storage medium, a deleting device that deletes data recorded on the storage medium, and the like. The storage device 908 stores programs executed by the CPU 901, various data, various data acquired from the outside, and the like.

### (Drive 909)

The drive 909 is a reader and writer for the storage medium and is built in or externally attached to the control device 40. The drive 909 reads information recorded on a mounted magnetic disk, optical disk, magneto-optical disk, or removable storage medium such as a semiconductor memory and outputs the information to the RAM 903. In addition, the drive 909 can also write information to the removable storage medium.

### (Connection Port 911)

The connection port 911 is a port for connecting an external connection device such as a universal serial bus (USB) port, an IEEE1394 port, a small computer system interface (SCSI), an RS-232C port, or an optical sound terminal.

### (Communication Device 913)

The communication device 913 is, for example, a communication interface formed by a communication device or the like for connecting to a network 920. The communication device 913 is, for example, a communication card for a wired or wireless local area network (LAN), long term evolution (LTE), Bluetooth (registered trademark), wireless USB (WUSB), or the like. Further, the communication device 913 may be a router for optical communication, a router for asymmetric digital subscriber line (ADSL), a modem for various communications, or the like. The communication device 913 can transmit and receive signals and the like to and from the Internet and other communication devices in accordance with a predetermined protocol such as TCP/IP.

Also, the network 920 is a wired or wireless transmission path for information transmitted from devices connected to the network 920. For example, the network 920 may include a public network such as the Internet, a telephone line network, a satellite communication network, various local area networks (LANs) including Ethernet (registered trademark), and a wide area network (WAN). In addition, the network 920 may include a dedicated network such as internet protocol-virtual private network (IP-VPN).

As described above, the hardware configuration example of the control device 40 according to the present embodiment has been described with reference to FIG. 17. Each of the above components may be realized by using a general-purpose member or may be realized by hardware specialized for the function of each component. Therefore, it is possible to appropriately change the hardware configuration to be used according to the technical level at each time when the present embodiment is implemented.

### 5. Summary

As described above, the medical device 100 according to the embodiment of the present disclosure includes the medical instrument 110 connected to the drive unit 150 via the power transmission mechanism and driven by the drive unit 150. In addition, the medical device 100 includes the pipe 120 having the hollow portion 91 through which the power transmission mechanism is inserted. Also, the medical device 100 includes the pipe 130 having the hollow portion 92 through which the pipe 120 is inserted into the hollow portion 92. Further, the medical device 100 includes the coupling portion 140 that couples the pipe 120 and the pipe 130. Furthermore, the medical device 100 includes the sensor unit 160 that measures vibrations related to the space 93 between the outer wall 121 of the pipe 120, the inner wall 131 of the pipe 130, and the coupling portion 140 on the medical instrument 110 side.

By having the above-described configuration, the medical device 100 can make it difficult to transmit the driving sounds generated by driving the drive unit 150 to the sensor unit 160. This makes it difficult for the sensor unit 160 to measure the driving sounds, which are vibration noise.

Therefore, it is possible to provide a new and improved medical device, a medical device component, and a master-slave system in which vibration noise transmitted to the sensor can be reduced.

Although the preferred embodiments of the present disclosure have been described in detail with reference to the accompanying figures, the technical scope of the present disclosure is not limited to such examples. It is clear that a person having ordinary knowledge in the technical field of the present disclosure may come up with various modified examples or changed examples within the scope of the technical ideas set forth in the claims, which are naturally understood to be within the technical scope of the present disclosure.

For example, each device described herein may be realized as a single device, or part or all of it may be realized as a separate device. For example, the slave device 10, the master device 20, the output device 30, and the control device 40 shown in FIG. 1 may be realized as independent devices. Further, the control device 40 shown in FIG. 1 may be realized as a server device connected to the slave device 10, the master device 20, and the output device 30 via a network or the like. Also, the control device 40 may be provided in at least one of the slave device 10 and the master device 20.

Further, a series of processes performed by each device described in the present specification may be realized by using software, hardware, or a combination of software and hardware. The programs constituting the software are stored in advance in, for example, recording media (non-transitory media) provided inside or outside each device. In addition, each program is read into RAM at the time of execution by a computer and executed by a processor such as a CPU.

Further, the effects described herein are merely explanatory or exemplary and are not limited. That is, the techniques according to the present disclosure may achieve other effects apparent to those skilled in the art from the description herein in addition to or in place of the above effects.

Further, the following configurations also belong to the technical scope of the present disclosure.

(1)

A medical device including:

a medical instrument that is connected to a drive unit via a power transmission mechanism and driven by the drive unit;

a first structural member having a first hollow portion through which the power transmission mechanism is inserted;

a second structural member having a second hollow portion through which the first structural member is inserted;

a third structural member that couples the first structural member and the second structural member; and

a sensor unit that measures vibrations related to a space between an outer wall of the first structural member, an inner wall of the second structural member, and the third structural member, the space being on a medical instrument side.

(2)

The medical device according to the above (1), wherein the sensor unit is positioned on the drive unit side in the space.

(3)

The medical device according to the above (2), wherein the sensor unit is provided in the second structural member or the third structural member in the space.

(4)

The medical device according to any one of the above (1) to (3), wherein the medical device further includes a sensor unit different from the sensor unit, and the different sensor unit measures vibration generated by driving the drive unit.

(5)

The medical device according to any one of the above (1) to (3), wherein a sensor unit different from the sensor unit is further provided outside the medical device, and

the different sensor unit measures vibration generated outside the medical device.

(6)

The medical device according to the above (4) or (5), wherein the vibration measured by the different sensor is used for removing noise included in the vibration measured by the sensor.

(7)

The medical device according to the above (4), wherein the different sensor unit is provided on the same structural member as the structural member provided with the sensor unit.

(8)

The medical device according to any one of the above (1) to (7), further including a fourth structural member, wherein

the fourth structural member couples the first structural member to the second structural member on a side of the space closer to the medical instrument than the sensor unit.

(9)

The medical device according to any one of the above (1) to (8), wherein the vibration includes vibration propagating in the air.

(10)

The medical device according to any one of the above (1) to (9), wherein the vibration includes vibration propagating in the medical device.

(11)

The medical device according to any one of the above (1) to (10), wherein

the medical instrument is driven as the power transmission mechanism is driven, and

the power transmission mechanism is driven as the drive unit is driven.

(12)

The medical device according to any one of the above (1) to (11), wherein the power transmission mechanism includes a wire.

(13)

A medical device component including:

a second structural member having a second hollow portion through which a first structural member of a medical device is inserted, the medical device including a medical instrument that is connected to a drive unit via a power transmission mechanism and driven by the drive unit, and the first structural member having a first hollow portion through which the power transmission mechanism is inserted; a third structural member that couples the first structural member to the second structural member; and

a sensor unit that measures vibrations related to a space between an outer wall of the first structural member, an inner wall of the second structural member, and the third structural member, which is on a medical instrument side.

(14)

A master-slave system including:

a medical device; wherein the medical device including: a medical instrument that is connected to a drive unit via a power transmission mechanism and driven by the drive unit; a first structural member having a first hollow portion through which the power transmission mechanism is inserted;

a second structural member having a second hollow portion through which the first structural member is inserted; a third structural member that couples the first structural member to the second structural member; and a sensor unit that measures vibrations related to a space between an outer wall of the first structural member, an inner wall of the second structural member, and the third structural member, the space being on a medical instrument side;

a slave device provided with the medical device; and

a master device used to operate the slave device.

### REFERENCE SIGNS LIST

10 Slave device
12 Distal end portion
20 Master device
30 Output device
40 Control device
100 Medical device
110 Medical instrument
120 Pipe
130 Pipe
140 Coupling portion
150 Drive unit
152 Pulley sliding portion
154 Wire
156 Motor
160 Sensor unit
170 Fastening member
180 O-ring
200 Operating body
210 Force sensor
400 Communication unit
410 Control unit
412 Acquisition unit
414 Signal processing unit
416 Drive control unit
418 Output control unit
1000 Master-slave system

1. A medical device comprising:
a medical instrument that is connected to a drive unit via a power transmission mechanism and driven by the drive unit;
a first structural member having a first hollow portion through which the power transmission mechanism is inserted;
a second structural member having a second hollow portion through which the first structural member is inserted;
a third structural member that couples the first structural member and the second structural member; and
a sensor unit that measures vibrations related to a space between an outer wall of the first structural member, an inner wall of the second structural member, and the third structural member, the space being on a medical instrument side.

2. The medical device according to claim 1, wherein the sensor unit is positioned on a drive unit side in the space.

3. The medical device according to claim 2, wherein the sensor unit is provided in the second structural member or the third structural member in the space.

4. The medical device according to claim 1, wherein
the medical device further includes a sensor unit different from the sensor unit, and
the different sensor unit measures vibration generated by driving the drive unit.

5. The medical device according to claim 1, wherein
a sensor unit different from the sensor unit is further provided outside the medical device, and
the different sensor unit measures vibration generated outside the medical device.

6. The medical device according to claim 4, wherein the vibration measured by the different sensor is used for removing noise included in the vibration measured by the sensor.

7. The medical device according to claim 4, wherein the different sensor unit is provided on the same structural member as the structural member provided with the sensor unit.

8. The medical device according to claim 1, further comprising a fourth structural member, wherein
the fourth structural member couples the first structural member to the second structural member on a side of the space closer to the medical instrument than the sensor unit.

9. The medical device according to claim 1, wherein the vibration includes vibration propagating in the air.

10. The medical device according to claim 1, wherein the vibration includes vibration propagating in the medical device.

11. The medical device according to claim 1, wherein
the medical instrument is driven as the power transmission mechanism is driven, and
the power transmission mechanism is driven as the drive unit is driven.

12. The medical device according to claim 1, wherein the power transmission mechanism includes a wire.

13. A medical device component comprising:
a second structural member having a second hollow portion through which a first structural member of a medical device is inserted, the medical device including a medical instrument that is connected to a drive unit via a power transmission mechanism and driven by the drive unit, and the first structural member having a first hollow portion through which the power transmission mechanism is inserted;
a third structural member that couples the first structural member to the second structural member; and
a sensor unit that measures vibrations related to a space between an outer wall of the first structural member, an inner wall of the second structural member, and the third structural member, which is on a medical instrument side.

14. A master-slave system comprising:
a medical device, the medical device including: a medical instrument that is connected to a drive unit via a power transmission mechanism and driven by the drive unit; a first structural member having a first hollow portion through which the power transmission mechanism is inserted; a second structural member having a second hollow portion through which the first structural member is inserted; a third structural member that couples the first structural member to the second structural member; and a sensor unit that measures vibrations related to a space between an outer wall of the first structural member, an inner wall of the second structural member, and the third structural member, the space being on a medical instrument side;
a slave device provided with the medical device; and
a master device used to operate the slave device,
